(19) United States
(12) Patent Application Publication
(10) Pub. No.: US 2008/0044882 A1
(43) Pub. Date: Feb. 21, 2008

(54) Oxidation by Hydrogen Peroxide

(75) Inventors: Hugh Allen Oliver Hill, Oxford (GB); Luet Lok Wong, Oxford (GB); Barry Dean Fleming, Clayton (AU)

Correspondence Address: FULBRIGHT & JAWORSKI L.L.P, 2200 ROSS AVENUE, SUITE 2800, DALLAS, TX, 75201-2784, US

(73) Assignee: ISIS INNOVATION LIMITED, OXFORD UNITED KINGDOM 0x2 7SG (GB)

(21) Appl. No.: 10/590,426

(22) PCT Filed: Feb. 23, 2005
(86) PCT No.: PCT/GB05/00674

§ 371 Date: May 8, 2007

(30) Foreign Application Priority Data
Feb. 23, 2004 (GB) 0403992.1

(51) Int. Cl. C12N 9/08; C12N 9/04

(52) U.S. Cl. 435/192; 435/190

(57) ABSTRACT

The present invention provides a method of carrying out an oxidation reaction catalysed by a monooxygenase enzyme and using hydrogen peroxide as an oxidant, in which reaction a low level of oxidation damage of the monooxygenase occurs, said method comprising producing the hydrogen peroxide simultaneously with the oxidation reaction, wherein the hydrogen peroxide is produced at a rate less than or equal to the rate at which it is used in the reaction or said method comprising carrying out the reaction in the presence of an H₂O₂ or hydroxyl radical sequestering agent that controls the H₂O₂ or hydroxyl radical concentration.

### FIELD OF THE INVENTION

The invention relates to a method of carrying out an oxidation reaction.

### BACKGROUND OF THE INVENTION

Monooxygenase enzymes catalyse the oxidation of a very wide range of substrates. In order to catalyse the reaction, a monooxygenase enzyme generally requires a cofactor and at least one electron-transfer partner protein (reductase). However, monooxygenase enzymes are capable of using hydrogen peroxide (H₂O₂) as an oxidizing agent because it acts as a source of dioxygen and two electrons. The use of H₂O₂ to drive oxidation reactions is known as the “peroxide shunt”.

### SUMMARY OF THE INVENTION

Monooxygenase enzymes generally have a high Kₘ for H₂O₂, (such as about 20 mM) in comparison to peroxidase enzymes. As a result, high concentrations of H₂O₂ are required for appreciable levels of activity of a monooxygenase enzyme when the oxidation reaction is performed using the peroxide shunt. For example, the initial rate of monooxygenase activity using 50 mM H₂O₂ is far below that when the natural co-factor, NAD(P)H, is used as with the physiological electron-transfer partners.

The invention provides a more efficient method of carrying out an oxidation reaction using the peroxide shunt by reducing the oxidative damage that occurs to the monooxygenase enzyme by not allowing excess levels of H₂O₂ to be present whilst the reaction is carried out.

Simultaneous production of H₂O₂ at a rate less than or equal to the rate at which it is used in an oxidation reaction catalysed by monooxygenase results in improved efficiency of the oxidation reaction and an increase in the product yield. Various methods may be used to produce H₂O₂ at the required rate, such as use of an electrochemical reaction, an enzyme or a precursor.

Accordingly, the present invention provides a method of carrying out an oxidation reaction catalysed by a monooxygenase enzyme and using hydrogen peroxide as an oxidant, in which reaction a low level of oxidation damage of the monooxygenase occurs, said method comprising producing the hydrogen peroxide simultaneously with the oxidation reaction, wherein the hydrogen peroxide is produced at a rate less than or equal to the rate at which it is used in the reaction.

The present invention also provides a method of carrying out an oxidation reaction catalysed by a monooxygenase enzyme and using hydrogen peroxide as an oxidant, in which reaction a low level of oxidation damage of the monooxygenase occurs, said method comprising carrying out the reaction in the presence of an H₂O₂ or hydroxyl radical sequestering agent that controls the H₂O₂ or hydroxyl radical concentration.

### DESCRIPTION OF THE SEQUENCES

SEQ ID NO: 1 shows the nucleotide sequence of cytochrome P450Cam from Pseudomonas putida.

SEQ ID NO: 2 shows the amino acid sequence of cytochrome P450Cam from Pseudomonas putida.

SEQ ID NO: 3 shows the nucleotide sequence of cytochrome P450BM-3 from Bacillus megaterium.

SEQ ID NO: 4 shows the amino acid sequence of cytochrome P450 BM-3 from Bacillus megaterium. The first 472 amino acid residues form the heme domain. The last 585 amino acid residues form the reductase domain. All 1048 amino acid residues form the holoenzyme.

The convention in the art, which is adopted herein, is to refer to a mutant with reference to the native amino acid residue at a position in the sequence, followed by the amino acid at that position in the mutant, e. g., F87 refers to the phenylalanine at position 87 in the wild-type sequence, and F87A refers to the phenylalanine at position 87 in the wild-type sequence which has been changed to alanine in the variant. The numbering of the amino acid residues starts with the amino acid residue following the initial methionine residue.

Mutants used in Examples were F87A (single mutation; SEQ ID NOs: 5 and 6) and F87V L188Q A74G (triple mutation; SEQ ID NOs: 7 and 8).

SEQ ID NO: 5 shows the amino acid sequence of the F87A mutant of cytochrome P450BM-3 from Bacillus megaterium.

SEQ ID NO: 6 shows the nucleotide sequence of of the F87A mutant of cytochrome P450BM-3 from Bacillus megaterium.

SEQ ID NO: 7 shows the amino acid sequence of the F87V L188Q A74G mutant of cytochrome P450BM-3 from Bacillus megaterium.

SEQ ID NO: 8 shows the nucleotide sequence of of the F87V L188Q A74G mutant of cytochrome P450BM-3 from Bacillus megaterium.

SEQ ID NO: 9 shows the nucleotide sequence of subunit 1 of B-276 alkene epoxidase from Nocardia coralline.

SEQ ID NO: 10 shows the amino acid sequence of subunit 1 of B-276 alkene epoxidase from Nocardia coralline.

SEQ ID NO: 11 shows the nucleotide sequence of subunit 2 of B-276 alkene epoxidase from Nocardia coralline.

SEQ ID NO: 12 shows the amino acid sequence of subunit 2 of B-276 alkene epoxidase from Nocardia coralline.

SEQ ID NO: 13 shows the nucleotide sequence of the alpha subunit of Py2 alkene monooxygenase from Xanthobacta sp.

SEQ ID NO: 14 shows the amino acid sequence of the alpha subunit of Py2 alkene monooxygenase from Xanthobacta sp.

SEQ ID NO: 15 shows the nucleotide sequence of the beta subunit of Py2 alkene monooxygenase from Xanthobacta sp.

SEQ ID NO: 16 shows the amino acid sequence of the beta subunit of Py2 alkene monooxygenase from Xanthobacta sp.

SEQ ID NO: 17 shows the nucleotide sequence of the gamma subunit of Py2 alkene monooxygenase from Xanthobacta sp.

SEQ ID NO: 18 shows the amino acid sequence of the gamma subunit of Py2 alkene monooxygenase from Xanthobacta sp.

SEQ ID NO: 19 shows the nucleotide sequence of the alpha subunit of soluble methane monooxygenase from Methylococcus capsulatas.

SEQ ID NO: 20 shows the amino acid sequence of the alpha subunit of soluble methane monooxygenase from Methylococcus capsulatas.

SEQ ID NO: 21 shows the nucleotide sequence of the beta subunit of soluble methane monooxygenase from Methylococcus capsulatas.

SEQ ID NO: 22 shows the amino acid sequence of the beta subunit of soluble methane monooxygenase from Methylococcus capsulatas.

SEQ ID NO: 23 shows the nucleotide sequence of the gamma subunit of soluble methane monooxygenase from Methylococcus capsulatas.

SEQ ID NO: 24 shows the amino acid sequence of the gamma subunit of soluble methane monooxygenase from Methylococcus capsulatas.

SEQ ID NO: 25 shows the nucleotide sequence of GPo1 alkane hydroxylase (AlkB gene) from Pseudomonas oleovorans.

SEQ ID NO: 26 shows the amino acid sequence of GPo1 alkane hydroxylase from Pseudomonas oleovorans.

SEQ ID NO: 27 shows the nucleotide sequence of the alpha subunit of toluene 2-monooxygenase from Burkholderia cepacia.

SEQ ID NO: 28 shows the amino acid sequence of the alpha subunit of toluene 2-monooxygenase from Burkholderia cepacia.

SEQ ID NO: 29 shows the nucleotide sequence of the beta subunit of toluene 2-monooxygenase from Burkholderia cepacia.

SEQ ID NO: 30 shows the amino acid sequence of the beta subunit of toluene 2-monooxygenase from Burkholderia cepacia.

SEQ ID NO: 31 shows the nucleotide sequence of the gamma subunit of toluene 2-monooxygenase from Burkholderia cepacia.

SEQ ID NO: 32 shows the amino acid sequence of the gamma subunit of toluene 2-monooxygenase from Burkholderia cepacia

SEQ ID NO: 33 shows the nucleotide sequence of phenol hydroxylase (pheA) gene from Bacillus stearothermophilus.

SEQ ID NO: 34 shows the amino acid sequence of phenol hydroxylase gene from Bacillus stearothermophilus.

SEQ ID NO: 35 shows the nucleotide sequence of stearoyl-ACP desaturase from Helianthus annuus.

SEQ ID NO: 36 shows the amino acid sequence of stearoyl-ACP desaturase from Helianthus annuus.

### DETAILED DESCRIPTION OF THE INVENTION

It is to be understood that this invention is not limited to particular embodiments. It is also to be understood that different applications of the disclosed methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a substrate” includes two or more substrates, reference to “an enzyme” includes reference to two or more enzymes, and the like.

All publications, patents and patent applications cited herein, whether supra or infra, are hereby incorporated by reference in their entirety.

The methods of the invention enable the oxidation of a variety of substrates. Such substrates include, but are not limited to, alkanes, aromatic compounds, terpenoid compounds, alkenes and fatty acids.

Suitable alkanes include, but are not limited to, methane, ethane, propane, butane, pentane, hexane, heptane, n-octane, n-nonane, n-decane, n-dodecane and n-hexadecane. The oxidation of alkanes produces alcohols. The oxidation of methane to methanol is technologically and economically very important. The medium-chain alcohols (e.g. n-octanol) are synthetic intermediates while the longer chain alcohols (e.g. n-dodecanol) are used for the synthesis of fatty acid derivatives.

Suitable aromatic compounds include, but are not limited to, benzene, toluene, xylene, chlorobenzene, phenol and substituents thereof. The phenolic and catecholic products are used in the synthesis of fragrance and flavour compounds.

Suitable terpenoid compounds include, but are not limited to, monoterpenes such as limonene, pinene, terpinene, and ocimene, sesquiterpenes such as valencene and aromadendrene and triterpenes which include the steroidal compounds. The products are intermediates for synthesis, fine fragrance and flavouring chemicals and pharmaceuticals.

Suitable alkenes include, but are not limited to, simple molecules such as propene, hex-1-ene, hex-2-ene, and styrene, and carbon-carbon double bonds in complex molecules. Selective epoxidation of alkenes to a single enantiomer is very important in synthesis. Optically pure propene oxide and styrene oxide are very useful intermediates in synthesis.

Hydroxylated fatty acids are precursors to polymers.

### Monooxygenase Enzyme

The enzyme used to carry out an oxidation reaction according to the invention is a monooxygenase enzyme. A person skilled in the art can determine whether an enzyme is a monooxygenase enzyme using standard techniques in the art. Typically, the prosthetic groups may be characterised using protein crystallography, especially for non-heme iron enzymes because they generally do not have chromophores. Otherwise, a person skilled in the art will typically use sequence alignment, looking for conserved motifs such as the active site, and iron content as well as subunit composition.

The monooxygenase enzyme preferably has a Kₘ for H₂O₂ of at least 15 nM, at least 20 nM, at least 25 nM, at least 30 nM, at least 35 nM, at least 40 nM, at least 45 nM or at least 50 nM.

Examples of monooxygenase enzymes include, but are not limited to, cytochrome P450 monooxygenases and non-heme di-iron monooxygenase enzymes. Suitable non-heme di-iron monooxygenase enzymes include, but are not limited to methane monooxygenase (Colby et al., Biochem. J., 1977; 165: 395-402; Dalton, Adv. Appl. Microbiol., 1980; 26: 71-87; Fox et al., J. Biol. Chem., 1989; 264: 10023-10033; Fox et al., Methods Enzymol., 1990; 188: 191-202; McDonald et al., Appl. Environ. Microbiol., 1997; 63: 1898-1904), alkane hydroxylase (van Beilen et al., Enzyme Microb. Technol., 1994; 16: 904-911), toluene monooxygenase (Luykx et al, Biochem. Biophys. Res. Commun., 2003; 312: 373-379; Pikus et al., Biochemistry, 1996; 35: 9106-9119; Newman & Wackett, Biochemistry, 1995; 34: 14066-14076), alkene monooxygenase (Gallagher et al., Eur. J. Biochem., 1997; 247: 635-641; Lange & Que, Curr. Opin. Chem. Biol., 1998; 2: 159-172; Zhou et al., FEBS Lett., 1998; 430: 181-185), phenol monooxygenase (Divari et al., Eur. J. Biochem., 2003; 270: 2244-2253) and steroid desaturase (Shanklin et al., Biochemistry, 1994; 33: 12787-12794). The non-heme di-iron monooxygenase enzymes are typically of eukaryotic or prokaryotic origin and preferably of bacterial, fungal, yeast, plant or animal origin. Preferred sequences are shown in SEQ ID NOs: 1 to 36.

The enzyme used in the methods of the invention is preferably a cytochrome P450 enzyme, typically of eukaryotic or prokaryotic origin. Cytochrome P450 monooxygenases are typically characterised by a 446-450 nm heme Soret band for the ferrous-carbon monoxide complex. The enzyme is generally of bacterial, fungal, yeast, plant or animal origin, and thus may be from a bacterium of the genus Pseudomonas. The enzyme may be a naturally-occurring form of P450, such as P450_{cam}, P450_{BM-3} from Bacillus megaterium, P450ₜₑᵣₚ from Pseudomonas sp, P450_{eryF} from Saccharopollyspora erythraea and also P450 105 D1 (CYP105) from Streptomyces griseus strains.

Alternatively, the enzyme may be a mutant of a naturally-occurring form of P450. The mutants retain the essential biological activity of the naturally-occurring enzyme, namely the ability to catalyse an oxidation reaction using H₂O₂. The mutant may have one or more mutations in the active site of the enzyme.

An amino acid ‘in the active site’ is one which lines or defines the site in which the substrate is bound during catalysis or one which lines or defines a site through which the substrate must pass before reaching the catalytic site. Therefore such an amino acid typically interacts with the substrate during entry to the catalytic site or during catalysis. Such an interaction typically occurs through an electrostatic interaction (between charged or polar groups), hydrophobic interaction, hydrogen bonding or van der Waals forces.

The amino acids in the active site can be identified by routine methods to those skilled in the art. These methods include labelling studies in which the enzyme is allowed to bind a substrate which modifies (‘labels’) amino acids which contact the substrate. Alternatively the crystal structure of the enzyme with bound substrate can be obtained in order to deduce the amino acids in the active site.

The monooxygenase enzyme may have 1, 2, 3, 4, 5 to 10, 10 to 20 or more other mutations, such as substitutions, insertions or deletions. Amino acid substitutions may be made to the amino acid sequence of a naturally-occurring enzyme, for example from 1, 2, 3, 4 or 5 to 10, 20 or 30 substitutions. Conservative substitutions may be made, for example, according to Table 1. Amino acids in the same block in the second column and preferably in the same line in the third column may be substituted for each other:

TABLE 1
Conservative amino acid substitutions

| | | |
|---|---|---|
| NON-AROMATIC | Non-polar | G A P |
| | | I L V |
| | Polar - uncharged | C S T M |
| | | N Q |
| | Polar - charged | D E |
| | | H K R |
| AROMATIC | | H F W Y |

The mutations may be in the active site or outside the active site. Typically the mutations are in the ‘second sphere’ residues which affect or contact the position or orientation of one or more of the amino acids in the active site. The insertion is typically at the N and/or C terminal and thus the enzyme may be part of a chimeric protein. The deletion typically comprises the deletion of amino acids which are not involved in catalysis, such as those outside the active site (thus the enzyme is a mutated fragment of a naturally occurring enzyme). The monooxygenase enzyme may thus comprise only those amino acids which are required for oxidation activity.

The mutation in the active site typically alters the position and/or conformation of the substrate when it is bound in the active site. The mutation may make the site on the substrate which is to be oxidized more accessible to the heme group. Thus the mutation may be a substitution to an amino acid which has a smaller or larger, or more or less polar, side chain.

The mutations typically increase the stability of the protein, or make it easier to purify the protein. They typically prevent the dimerisation of the protein, typically by removing cysteine residues from the protein (e.g. by substitution of cysteine at position 334 of P450_{cam}, or at an equivalent position in a homologue, preferably to alanine). They typically allow the protein to be prepared in soluble form, for example by the introduction of deletions or a poly-histidine tag, or by mutation of the N-terminal membrane anchoring sequence. The mutations typically inhibit protein oligomerisation, such as oligomerisation arising from contacts between hydrophobic patches on protein surfaces.

The mutations may affect the manner in which the enzyme utilises H₂O₂ and thereby improve the efficiency of the reaction. For example, replacement of all the methionine residues of the heme domain of P450_{BM-3} with norleucine results in a two-fold increase in the peroxygenase activity of the enzyme (Cirino et al., Biotechnol. Bioeng., 2003; 83(6): 729-734). Furthermore, direct evolution studies to find mutants of enzymes more resistant to peroxide have been reported (Cirino & Arnold, Angew. Chem. Int. Ed., 2003; 42: 3299-3301).

Thus the mutant enzyme is typically at least 70% homologous to a naturally occurring enzyme on the basis of amino acid identity.

A mutant protein (i.e. described as being a mutant of another protein) mentioned herein is typically at least 70% homologous to the relevant protein or at least 80 or 90% and more preferably at least 95%, 97% or 99% homologous thereto over at least 20, preferably at least 30, for instance at least 40, 60 or 100 or more contiguous amino acids. The contiguous amino acids may include the active site. This homology may alternatively be measured not over contiguous amino acids but over only the amino acids in the active site.

Homology can be measured using known methods. For example the UWGCG Package provides the BESTFIT program which can be used to calculate homology (for example used on its default settings) (Devereux et al (1984) Nucleic Acids Research 12, p 387-395). The PILEUP and BLAST algorithms can be used to calculate homology or line up sequences (typically on their default settings), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

Mutants include fragments of the above-mentioned sequences. Such fragments retain monooxygenase activity. Fragments may be at least 300, at least 400 or at least 450 amino acids in length. Such fragments may be used to produce chimeric enzymes as described in more detail below.

Mutants also include chimeric proteins comprising fragments or portions of a naturally-occurring enzyme. One or more amino acids may be alternatively or additionally added to the polypeptides described above. An extension may be provided at the N-terminus or C-terminus of the naturally-occurring enzyme or variant or fragment thereof. The extension may be quite short, for example from 1 to 10 amino acids in length. Alternatively, the extension may be longer. A carrier protein may be fused to an amino acid sequence described above. A fusion protein incorporating one of the enzymes described above can thus be used in the invention.

The naturally-occurring enzyme or mutant thereof may also be chemically-modified. A number of side chain modifications are known in the art and may be made to the side chains of the enzymes discussed above. Such modifications include, for example, glycosylation, phosphorylation, modifications of amino acids by reductive alkylation by reaction with an aldehyde followed by reduction with NaBH₄, amidination with methylacetimidate or acylation with acetic anhydride. The modification is preferably glycosylation.

The mutations discussed herein are generally introduced into the enzyme by using methods known in the art, such as site directed mutagenesis of the enzyme, PCR and gene shuffling methods or by the use of multiple mutagenic oligonucleotides in cycles of site-directed mutagenesis. Thus the mutations may be introduced in a directed or random manner. The mutagenesis method thus produces one or more polynucleotides encoding one or more different mutants. Typically a library of mutant oligonucleotides is produced which can be used to produce a library of mutant enzymes.

The enzyme may be made synthetically or by recombinant means using methods known in the art. The amino acid sequence of the monooxygenase enzyme may be modified to include non-naturally occurring amino acids or to increase the stability of the enzyme. When the enzyme is produced by synthetic means, such amino acids may be introduced during production. The proteins or peptides may also be modified following either synthetic or recombinant production.

The enzyme may also be produced using D-amino acids. In such cases the amino acids will be linked in reverse sequence in the C to N orientation. This is conventional in the art for producing such proteins or peptides.

The enzyme may be produced in a cell by in situ expression of the polypeptide from a recombinant expression vector. The expression vector optionally carries an inducible promoter to control the expression of the polypeptide. The enzyme may be produced in large scale following purification by any protein liquid chromatography system after recombinant expression. Preferred protein liquid chromatography systems include FPLC, AKTA systems, the Bio-Cad system, the Bio-Rad BioLogic system and the Gilson HPLC system.

### Oxidation Reaction

The methods of the invention concerns carrying out a high efficiency oxidation reaction catalysed by a monooxygenase enzyme. A high efficiency oxidation reaction is a reaction that occurs without an appreciable reduction in the enzyme turnover or product yield or inactivation of the monooxygenase enzyme. Preferably, the monooxygenase enzyme displays at least 70%, at least 80%, at least 90%, at least 95% or 100% of the activity shown at the beginning of the reaction after 1 hour, 2 hours, 6 hours, 12 hours, 1 day, 2 days or 5 days.

Typically the methods of the invention are carried out in vitro, such as in a cell free system.

The reaction is driven by the “peroxide shunt”. The reaction of the invention is carried out in the presence of the monooxygenase enzyme (a), the substrate (b) and H₂O₂ (c). The reaction is typically performed in aerobic conditions and does not require any cofactors. The production of (c) is discussed in more detail below. In this system the flow of electrons is typically: (c)→(a)→(b).

In the methods the concentration of (a) and (b) is typically from 10⁻⁸ to 10⁻² M, preferably from 10⁻⁶ to 10⁻⁴ M. Typically the ratio of concentrations of (a): (b) is from 0.1:10 to 1:10, preferably from 1:0.5 to 1:2, or from 1:0.8 to 1:1.2. Preferably, the concentration of (b) is greater than the concentration of (a). The preferred concentration of (a) is that which when reacted with substrate will generate sufficient product to be detected by available analytical methods e.g. GC, HPLC. This is typically of the order of μM quantities.

Generally the methods are carried out at a temperature and/or pH at which the monooxygenase enzyme is functional, such as when the enzyme has at least 20%, 50%, 80% or more of peak activity. Typically the pH is from 2 to 11, such as from 5 to 9 or from 6 to 8, preferably from 7 to 7.8 or 7.4. The pH can be maintained using a suitable buffering agent such as phosphate or acetate based systems. Typically the temperature is from 0 to 80° C., such as from 25 to 75° C., from 30 to 60° C. or from 50° C. to 80° C. Preferably, the temperature is from 20 to 40° C.

Typically in the methods at least 20 turnovers/min occur, such as at least 50, 100, 200, 300, 500 or more turnovers (turnover is measured as nanomoles of product formed per nanomole of enzyme).

Typically, the rate of H₂O₂ production is less than or equal to 1, 2 or 3 μg per min per mg of monooxygenase enzyme. Typically, the concentration of H₂O₂ throughout the reaction is less than or equal to 0.1, 0.5 or 1 mM. Typically, the reaction continues for at least 60 minutes, at least 240 minutes, at least 6 hours or at least 10 hours.

The methods of the invention may be carried out in the monooxygenase substrate if it is a liquid under the reaction conditions. The methods of the invention may also be conducted in a solvent. Suitable solvents include, but are not limited to, water, aqueous buffer solutions mixed water/organic and aqueous buffer/organic solvent systems. Preferably, the organic solvent is a hydrocarbon such as hexane, benzene, acetonitrile, lower aliphatic alcohols, ketones and dioxane, dimethylformanide and dimethylsulphoxide and mixtures thereof. The solvent is typically one in which the reagants and products are highly soluble and one that maintains the stability and activity of the monooxygenase enzyme.

The reaction may be carried out in a homogenous system with all the components in solution. Typically, the monooxygenase enzyme and substrate are mixed together in a suitable solvent in a stirred tank reactor and the reaction is conducted in batch, semi-batch or continuous mode.

Alternatively, the monooxygenase enzyme may be immobilized on a suitable solid support, such as silica, prior to carrying out the method of the invention. An immobilized monooxygenase enzyme can be packed into a fixed bed reactor and the substrate passed over the enzyme. In one embodiment, the enzyme producing the H₂O₂ (discussed in more detail below) may be immobilized on the same or different material as the monooxygenase enzyme. Procedures for immobilizing enzymes are known in the art. Examples of such procedures include, but are not limited to, covalent coupling to insoluble organic or inorganic supports, entrapment in gels and adsorption to ion exchange resins or other adsorbent materials. (G. F. Bickerstaff ed., “Immobilization of Enzymes and Cells,” Humana Press, Totowa. N.J., 1997).

In a further embodiment, a membrane on the “entry” side admits the substrate slowly from the “reactant” side and then a hydrophilic membrane on the “exit” side allows hydrophilic compounds to flow out to the “product” side of the flow reaction cell. In this case the H₂O₂ may be generated outside the membrane and allowed to flow through the membrane to the mobile or immobile enzyme.

In one embodiment, H₂O₂ is preferably produced by one of the methods discussed in more detail below. In another embodiment, a H₂O₂ or hydroxyl radical sequestering agent is used to sequester excess H₂O₂ or hydroxyl radical during the oxidation reaction. The sequestering agent may be a chelating agent. In one embodiment, the chelating agent is EDTA. The EDTA inhibits production of the hydroxyl radical, for example, produced by the reaction of trace amounts of iron (or copper) with the H₂O₂.

### H₂O₂ Production by an Electrochemical Reaction

The H₂O₂ may be produced in the method of the invention by an electrochemical reaction. An electrochemical reaction is generally a means for introducing a current to a liquid, preferably a solution. An electrochemical reaction is typically an oxidation or reduction reaction that takes place at an electrode through which a current flows. An electrode is a solid capable of conducting electricity, typically carbon-based or metallic, leading to an external source or sink which is in contact with the liquid, preferably a solution. The electrode may be either positively charged (cathode) or negatively charged (anode). Two or more electrodes may form an electrochemical cell from which an external wire can lead from each electrode to an external electrical device. An oxidation or reduction reaction takes place at one electrode, while a redox reaction can take place either in an electrochemical cell or directly in the liquid.

Production of H₂O₂ using an electrochemical reaction is energy efficient. H₂O₂ is typically produced by the controlled electrochemical reduction of molecular oxygen to hydrogen peroxide. The surface area and the overpotential of the cathode are key considerations for the two-electron reduction of molecular oxygen to hydrogen peroxide. Typically, carbon-based cathodes are used and they may be modified with a compound known to lower the overpotential for this reaction. Electrode materials and modifiers which will perform this task effectively and efficiently are well known in the art. The reduction of O₂, and hence production of hydrogen peroxide, can typically be controlled by the potential applied to the cathode. The potential applied to the cathode will vary depending on the cathode and any modifications to the cathode made.

The electrochemical reaction used in the method of the invention may be the sonoelectrochemical reduction of dioxygen. This method is well known in the art (Compton et al., Electroanalysis, 1997; 9(7): 509-522).

### H₂O₂ Production by an Enzyme

The H₂O₂ may be produced in the method of the invention by an enzyme. The enzyme is preferably an oxidase. Examples of suitable oxidases include, but are not limited to, glucose oxidase (E.C. 1.1.3.4), secondary-alcohol oxidase (E.C. 1.1.3.18), methanol oxidase (E.C. 1.1.3.31), oxalate oxidase (E.C. 1.2.3.4), aryl-aldehyde oxidase (E.C. 1.2.3.9), carbon monoxide oxidase (E.C. 1.2.3.10), amine oxidase (E.C. 1.4.3.4), ethanolamine oxidase (E.C. 1.4.3.8), nitroethane oxidase (E.C. 1.7.3.1) and sulfite oxidase (E.C. 1.8.3.1). Glucose oxidase (E.C. 1.1.3.4) catalyzes the conversion of D-glucose to D-glucono-1,5-lactone and H₂O₂. Secondary-alcohol oxidase (E.C. 1.1.3.18) catalyzes the conversion of a secondary alcohol to a ketone and H₂O₂. Methanol oxidase (E.C. 1.1.3.31) catalyzes the conversion of methanol to formaldehyde and H₂O₂. Oxalate oxidase (E.C. 1.2.3.4) catalyzes the conversion of oxalate to carbon dioxide and H₂O₂. Aryl-aldehyde oxidase (E.C. 1.2.3.9) catalyzes the conversion of an aromatic aldehyde to an aromatic acid and H₂O₂. Carbon monoxide oxidase (E.C. 1.2.3.10) catalyzes the conversion of CO and H₂O to carbon dioxide and H₂O₂. Amine oxidase (E.C. 1.4.3.4) catalyzes the conversion of RCH₂NH₂ and H₂O to RCHO and NH₃ and H₂O₂. Ethanolamine oxidase (E.C. 1.4.3.8) catalyzes the conversion of ethanolamine and H₂O to glycolaldehyde and H₂O₂. Nitroethane oxidase (E.C. 1.7.3.1) catalyzes the conversion of nitroethane and H₂O to acetaldehyde and H₂O₂. Sulfite oxidase (E.C. 1.8.3.1) catalyzes the conversion of sulfite and H₂O₂ to sulfate and H₂O₂. The oxidase may be purchased commercially (e.g., glucose oxidase). Alterantively, the oxidase can be extracted from known microorganisms using procedures known in the art.

The substrate for the oxidase will be well known in the art. In addition to the substrate, the reaction to produce H₂O₂ will also involve water. Typically, a H₂O₂-activating metal is also included in the reaction. Suitable metals include, but are not limited to, cerium, chromium, cobalt, copper, iron, manganese, molybdenum, silver, titanium, tungsten, vanadium and mixtures thereof. Metallosilicates containing the above metals can be prepared and used in the method of the invention. The procedure for producing such metallosilicates in known in the art (Neumann et al., Journal of Catalysis, 1997; 166: 206-127). The metallosilicate is preferably tetrahedrally coordinated titanium such as silicalite-1 (TS-1), silicalite-2 (TS-2), zeolite-beta, silicon analogs of ZSM-48 and MCM-4 1. (Murugavel and Roesky, Angew. Chem. Int. Ed. Engl., 1997; 36(5): 477-479).

In a preferred embodiment of the invention, the metal-containing solid or metallosilicate is used as a support upon which the H₂O₂-producing enzyme is immobilized. In another preferred embodiment, the monooxygenase enzyme is also immobilized on the same or different metallosilicate.support.

Preferably, the oxidase is first mixed with the other reaction components and then the reaction is initiated by addition of the oxidase substrate. For example, the monooxygenase enzyme, monoxygenase enzyme substrate and oxidase are all mixed and then the oxidase enzyme is added. In a preferred embodiment, P450BM3, octane and glucose oxidase are mixed together and then glucose added. Control of H₂O₂ generation can typically be accomplished by controlling the rate at which the oxidase substrate is added.

### H₂O₂ Production by a Precursor

The H₂O₂ may be produced in the method of the invention by a precursor. The generation of H₂O₂ by the addition of a precursor to water is well known in the art. Precursors include, but are not limited to, salts of perborate, salts of percarbonate, salts of perphosphate and peroxynitrite. Preferred precursors are sodium salts. The H₂O₂-producing properties of the precursor may be enhanced by using a compound such as tetraacetylethylenediamine. The amount of precursor added to the solution containing the monoxygenase enzyme and substrate is such to maximise the enzymatic reaction with the substrate and to minimise the deactivation of the enzyme by H₂O₂. Preferably the concentration of H₂O₂ produced does not exceed the Kₘ value for the enzyme but is sufficient to generate the enzyme reactive species.

### EXAMPLES

### Example 1

In this experiment, octane was reacted with electrochemically generated H₂O₂ in the presence of P450_{BM3} heme domain. The experiment was performed at room temperature with a three-electrode configuration in a 100 mL glass beaker. The reticulated vitreous carbon (RVC) cathode, platinum gauze anode and Ag/AgCl reference electrode were contained in the one vessel. The RVC cathode was briefly immersed in a 1 mM 2-aminoanthraquinone ethanolic solution before being removed and allowed to dry in air. The reaction solution contained aqueous Tris buffer (50 mM, pH 7.4) saturated with oxygen, 0.2 M KCl, 0.5 mM octane, and 3 μM P450_{BM3} F87V L188Q A74G heme domain. The reaction solution was stirred to equilibrate (5-10 minutes) and then a potential of −0.55 V vs Ag/AgCl was applied for 2 hours and the solution stirred continuously throughout. GC analysis revealed the presence of the solvent chloroform, octane, 2-, 3- and 4-octanol and the internal standard 1-nonanol. The relative proportion of 2, 3 & 4-octanol was 1:1.1:0.7. The total concentration of octanols formed was 141 μM, representing a turnover per enzyme of 47.

A similar experiment was performed with 1.43 μM wild-type P450_{BM3} heme domain. The total concentration of octanols formed was 8.4 μM, representing a turnover per enzyme of 6. The relative proportion of 2, 3 & 4-octanol in this case was 1:1.7:2.0.

### Example 2

In this experiment, octane was reacted with enzymatically generated H₂O₂ in the presence of P450_{BM3} holoenzyme. Into a glass vial was added a solution (total volume 5 mL) consisting of aqueous Tris buffer (50 mM, pH 7.4), 0.5 mM octane, 1.6 μM P450_{BM3} F87V L188Q A74G holoenzyme and glucose oxidase (1.5 U). After equilibration (5 mins), the reaction was initiated by addition of glucose (1×10⁻⁶ moles). Successive additions of glucose (1×10⁻⁶ moles) were made every 5 minutes up to 1 hour (total of 12 additions equivalent to 1.2×10⁻⁵ moles). The reaction was stirred continuously during this time and stopped after 1.5 hours. GC analysis revealed the presence of the solvent chloroform, octane, 2-, 3- and 4-octanol and the internal standard 1-nonanol. The relative proportion of 2, 3 & 4-octanol was 1:1.1:0.8. The total concentration of octanols formed was 17 μM, representing a turnover per enzyme of 10.

### Example 3

In this experiment, octane was reacted with H₂O₂ derived from sodium perborate, in the presence of P450_{BM3} holoenzyme. Into a glass vial was added a solution (total volume 5 mL) consisting of aqueous Tris buffer (40 mM, pH 7.4), 0.5 mM octane, and 1.3 μM P450_{BM3} F87V L188Q A74G holoenzyme. After equilibration (5 mins), the reaction was initiated by addition of NaBO₃.4H₂O (1×10⁻⁴ moles) and stirred continuously for 1 hour. GC analysis revealed the presence of the solvent chloroform, octane, 2-, 3- and 4-octanol and the internal standard 1-nonanol. The relative proportion of 2, 3 & 4-octanol was 1:1.8:1.1. The total concentration of octanols formed was 77 μM, representing a turnover per enzyme of 59.

For Examples 1 to 3, no octanol products were observed when the P450 enzyme was absent from the solution.

### Example 4

In this experiment, pinene was reacted with H₂O₂ derived from sodium perborate, in the presence of P450_{BM3} heme domain. Into a glass vial was added a solution (total volume 5 mL) consisting of aqueous Tris buffer (40 mM, pH 7.4), 0.63 mM pinene, and 3.7 μM wild-type P450_{BM3} heme domain. After equilibration (5 mins), the reaction was initiated by addition of 7.8×10⁻⁶ moles NaBO₃.4H₂O and stirred continuously for 1 hour. GC analysis revealed the presence of cis/trans 2,3-epoxides (32%), (+)-trans-verbenol (16%), (+)-cis-verbenol (6%), (+)-verbenone/(+)-myrtenol (13%), myrtenal (4%), as well as unidentified further oxidation products (29%). The total concentration of products formed was 80 μM, representing a turnover per enzyme of 22.

### Example 5

In this experiment, phenol monooxygenase is reacted with phenol in the presence of with H₂O₂ generated by sodium perborate. Into a glass vial is added a solution (total volume 5 mL) consisting of aqueous Tris buffer (40 mM, pH 7.4), 0.63 mM phenol, and 3.7 μM wild-type phenol monooxygenase. After equilibration (5 mins), the reaction is initiated by addition of 7.8×10⁻⁶ moles NaBO₃.4H₂O and stirred continuously for 1 hour. GC analysis reveals the presence of oxidation products.

### Example 6

In this experiment, P450_{BM3} is reacted with palmitic acid in the presence of H₂O₂ generated by glucose oxidase. Into a glass vial is added a solution (total volume 5 mL) consisting of aqueous Tris buffer (50 mM, pH 7.4), 0.5 mM palmitic acid, 1.6 μM P450_{BM3} holoenzyme and glucose oxidase (1.5 U). After equilibration (5 mins), the reaction is initiated by addition of glucose (1×10⁻⁶ moles). Successive additions of glucose (1×10⁻⁶ moles) are made every 5 minutes up to 1 hour (total of 12 additions equivalent to 1.2×10⁻⁵ moles). The reaction is stirred continuously during this time and stopped after 1.5 hours. GC analysis reveals the presence of oxidation products.

### Example 7

Plant CYP74C is reacted with 13 S-hydroperoxylinolenic acid to form the compound 3Z-hexenal (a fragrance). The H₂O₂ is generated by sodium perborate. Into a glass vial is added a solution (total volume 5 mL) consisting of aqueous Tris buffer (40 mM, pH 7.4), 0.63 mM 13 S-hydroperoxylinolenic acid, and 3.7 μM wild-type plant CYP74C. After equilibration (5 mins), the reaction is initiated by addition of 7.8×10⁻⁶ moles NaBO₃.4H₂O and stirred continuously for 1 hour. GC analysis reveals the presence of oxidation products.

1. A method of carrying out an oxidation reaction catalysed by a monooxygenase enzyme and using hydrogen peroxide as an oxidant, in which reaction a low level of oxidation damage of the monooxygenase occurs, said method comprising producing the hydrogen peroxide simultaneously with the oxidation reaction, wherein the hydrogen peroxide is produced at a rate less than or equal to the rate at which it is used in the reaction.

2. A method according to claim 1, wherein the monooxygenase enzyme has a Kₘ for H₂O₂ of at least 15 nM.

3. A method according to claim 1, wherein the monooxygenase enzyme is a P450 enzyme.

4. A method according to claim 1, wherein the rate of H₂O₂ production is less than or equal to 3 μg per mg of enzyme.

5. A method according to claim 1, wherein the concentration of H₂O₂ throughout the reaction is less than or equal to 1 mM.

6. A method according to claim 1, wherein the reaction continues for at least 240 minutes.

7. A method according to any one of the preceding claims 1, wherein the H₂O₂ is produced by an electrochemical reaction.

8. A method according to claim 1, wherein the H₂O₂ is produced by an enzyme reaction.

9. A method according to claim 8, wherein the enzyme is glucose oxidase.

10. A method according to claim 1, wherein the H₂O₂ is produced by a H₂O₂ precursor.

11. A method according to claim 10, wherein the H₂O₂ precursor is perborate, percarbonate or perphosphate.

12. A method preceding claim 1, wherein the substrate which is oxidised by the monooxygenase enzyme is an alkane, aromatic compound, terpenoid compound, alkene or fatty acid.

13-15. (canceled)

16. A method of carrying out an oxidation reaction catalysed by a monooxygenase enzyme and using hydrogen peroxide as an oxidant, in which reaction a low level of oxidation damage of the monooxygenase occurs, said method comprising carrying out the reaction in the presence of an H₂O₂ or hydroxyl radical sequestering agent that controls the H₂O₂ or hydroxyl radical concentration.

17. A method according to claim 16, wherein the sequestering agent is EDTA.


SEQUENCE LISTING (36 sequences)

SEQ ID NO: 1 - DNA, 1248 bp, Pseudomonas putida
  CDS (1)..(1248)
  misc_feature (4)..(4)
atgacgactgaaaccatacaaagcaacgccaatcttgcccctctgccaccccatgtgccagagcacctggtattcgacttcgacatgtacaatccgtcgaatctgtctgccggcgtgcaggaggcctgggcagttctgcaagaatcaaacgtaccggatctggtgtggactcgctgcaacggcggacactggatcgccactcgcggccaactgatccgtgaggcctatgaagattaccgccacttttccagcgagtgcccgttcatccctcgtgaagccggcgaagcctacgacttcattcccacctcgatggatccgcccgagcagcgccagtttcgtgcgctggccaaccaagtggttggcatgccggtggtggataagctggagaaccggatccaggagctggcctgctcgctgatcgagagcctgcgcccgcaaggacagtgcaacttcaccgaggactacgccgaacccttcccgatacgcatcttcatgctgctcgcaggtctaccggaagaagatatcccgcacttgaaatacctaacggatcagatgacccgtccggatggcagcatgaccttcgcagaggccaaggaggcgctctacgactatctgataccgatcatcgagcaacgcaggcagaagccgggaaccgacgctatcagcatcgttgccaacggccaggtcaatgggcgaccgatcaccagtgacgaagccaagaggatgtgtggcctgttactggtcggcggcctggatacggtggtcaatttcctcagcttcagcatggagttcctggccaaaagcccggagcatcgccaggagctgatcgagcgtcccgagcgtattccagccgcttgcgaggaactactccggcgcttctcgctggttgccgatggccgcatcctcacctccgattacgagtttcatggcgtgcaactgaagaaaggtgaccagatcctgctaccgcagatgctgtctggcctggatgagcgcgaaaacgcctgcccgatgcacgtcgacttcagtcgccaaaaggtttcacacaccacctttggccacggcagccatctgtgccttggccagcacctggcccgccgggaaatcatcgtcaccctcaaggaatggctgaccaggattcctgacttctccattgccccgggtgcccagattcagcacaagagcggcatcgtcagcggcgtgcaggcactccctctggtctgggatccggcgactaccaaagcggtataa

SEQ ID NO: 2 - protein, 415 aa, Pseudomonas putida
MTTETIQSNANLAPLPPHVPEHLVFDFDMYNPSNLSAGVQEAWAVLQESNVPDLVWTRCNGGHWIATRGQLIREAYEDYRHFSSECPFIPREAGEAYDFIPTSMDPPEQRQFRALANQVVGMPVVDKLENRIQELACSLIESLRPQGQCNFTEDYAEPFPIRIFMLLAGLPEEDIPHLKYLTDQMTRPDGSMTFAEAKEALYDYLIPIIEQRRQKPGTDAISIVANGQVNGRPITSDEAKRMCGLLLVGGLDTVVNFLSFSMEFLAKSPEHRQELIERPERIPAACEELLRRFSLVADGRILTSDYEFHGVQLKKGDQILLPQMLSGLDERENACPMHVDFSRQKVSHTTFGHGSHLCLGQHLARREIIVTLKEWLTRIPDFSIAPGAQIQHKSGIVSGVQALPLVWDPATTKAV

SEQ ID NO: 3 - DNA, 4957 bp, Bacillus megaterium
  CDS (1541)..(4690)
agatctttatgaagacatagctgcagaagaaaaagcaagagctacatatcaatggttaattgatatatcagatgatcccgatttaaacgacagcttacgatttttacgagaaagagagattgttcactcacagcggttccgcgaggccgtggagattttaaaagatgacagagacaggaagaaaatcttttaactagtaaaaaaacatcccccttggcgaatgcaaacgaaaggagggatgttttttgttgtgactgcgttgattatgcgctagaactgcagtgacaagaaacaacctttaatttcccttcaacatctttccaaactcgcgtataactgtattcacctccaatagattcaccggttgccagtgccccatttaacgctacttttgtaacggtaacggcaagttcttgaaacagtttaacttcttgttccaacacttccatgcccgctatatcaagactttttgaacgatgaacatttatatcttcttcttttgacaaccattgcccaaggtgattcacaaaaataagctcatctgaaagtaattcttctaatagctctatgttattagaaagcatggctgagcgaagcatttcttcgtattctataactcttgcttgattcatttttaatcctcctttacgccttgtgtaactcttttctatttccacgttgcttttcctttaaacttctttcattaataattcgtgctaaattatgttaatagaggggataagtggactaattttctgtaagcactaaatattctgaaatactctgttaattacctttaaatggtataaaattagaatgaaagaaccttttctttccacttttctagttatctttttactattaagatgcagttttttatacttgtaattgtagcggaatgaacgttcattccgtttttgaaaagaggtgataaagtggaatctactccaacaaaacaaaaagcgattttttctgcttcgcttctgctgtttgcagaaagagggtttgatgcaaccacgatgccaatgattgcagagaatgccaaagtaggagcaggaacaatttatcgctactttaaaaataaagaaagccttgtaaatgaattattccaacagcacgtaaacgagtttttacagtgcattgaaagcggtctggcaaacgagagagatggataccgagatgggtttcatcatatctttgaaggtatggtgacatttactaaaaaccatcctcgtgctcttggatttattaaaactcatagccaaggaacttttttaacagaagagagccgcttagcatatcaaaagctggtggaatttgtttgtacgttcttcagagaaggacaaaagcaaggtgtgattagaaatcttcctgaaaatgcgctaattgctattttatttggaagtttcatggaagtatatgaaatgattgaaaatgactacttatctttaactgatgaacttcttaccggtgtagaagagagtctgtgggcagcacttagcagacaatcatgaaacttaacaagtgaaagagggataacatgacaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgtacgtgattttgcaggagacgggttatttacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgcgttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctgggtaaattaaaaagaggctaggataaaagtagtttagttggttgaaggaagatccgaacgatgaatcgttcggatctttttattggtagagtaaacgtagatttcatctatttagtgacttgtagcggttgattggagggcaaggtgaagactccaatcaaccgcggtgtcacatgcaagccatacgaaattcatttctcccatttattcgtcttttgtccccacttaatttttatagcgccttaacgtttcttctgcgtgacagcagatct

SEQ ID NO: 4 - protein, 1049 aa, Bacillus megaterium [sequence omitted]

SEQ ID NO: 5 - DNA, 3147 bp, Artificial sequence
  Cytochrome P450BM-3 mutant
acaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcgcttaaatttgtacgtgattttgcaggagacgggttagctacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagctgcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgcgttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctgggtaa

SEQ ID NO: 6 - protein, 1048 aa, Artificial sequence
  Synthetic Construct
TIKEMPQPKTFGELKNLPLLNTDKPVQALMKIADELGEIFKFEAPGRVTRYLSSQRLIKEACDESRFDKNLSQALKFVRDFAGDGLATSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 7 - DNA, 3147 bp, Artificial sequence
  Cytochrome P450BM-3 mutant
acaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagggcttaaatttgtacgtgattttgcaggagacgggttagttacaagctggacgcatgaaaaaaattggaaaaaagcgcataatatcttacttccaagcttcagtcagcaggcaatgaaaggctatcatgcgatgatggtcgatatcgccgtgcagcttgttcaaaagtgggagcgtctaaatgcagatgagcatattgaagtaccggaagacatgacacgtttaacgcttgatacaattggtctttgcggctttaactatcgctttaacagcttttaccgagatcagcctcatccatttattacaagtatggtccgtgcactggatgaagcaatgaacaagcagcagcgagcaaatccagacgacccagcttatgatgaaaacaagcgccagtttcaagaagatatcaaggtgatgaacgacctagtagataaaattattgcagatcgcaaagcaagcggtgaacaaagcgatgatttattaacgcatatgctaaacggaaaagatccagaaacgggtgagccgcttgatgacgagaacattcgctatcaaattattacattcttaattgcgggacacgaaacaacaagtggtcttttatcatttgcgctgtatttcttagtgaaaaatccacatgtattacaaaaagcagcagaagaagcagcacgagttctagtagatcctgttccaagctacaaacaagtcaaacagcttaaatatgtcggcatggtcttaaacgaagcgctgcgcttatggccaactgctcctgcgttttccctatatgcaaaagaagatacggtgcttggaggagaatatcctttagaaaaaggcgacgaactaatggttctgattcctcagcttcaccgtgataaaacaatttggggagacgatgtggaagagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctggatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctgggtaa

SEQ ID NO: 8 - protein, 1048 aa, Artificial sequence
  Synthetic Construct
TIKEMPQPKTFGELKNLPLLNTDKPVQALMKIADELGEIFKFEAPGRVTRYLSSQRLIKEACDESRFDKNLSQGLKFVRDFAGDGLVTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKQQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 9 - DNA, 1032 bp, Nocardia corallina
  CDS (1)..(1032)
atgacgacagaggcgacggtggcccgaccggtggagctcgaaggtcaccggacattcacctggttcacgcccgccaggcgaaagccgacggagtacgagctctacaccgtgggtcaacagtccactccggacgagtggctgcatgtggactggccgctgcgcttcgacgacggccgcgccccgtgggaggaggagtcgagtgcggtacggacctcggagtggtcggcttaccgcgacccacaccaactgtggcagcgtccctacgtcagcacgtgcaaccaggaccagcaggccctcgcgcggctggtccccgtcctgaccatggggtcggcggcgatcacgcccatctggtcgcagaagatcctcgccaggtcctacgccgcctggccattcgtcgagtacgggctcttcctgagcctggcctacgccgtgcgccaggccatgtccgacacggtccagttcagcgtggtgttccaggccgtggaccgcatgcggctgctccaggacatcgtccaccacctggaccacctgcaggagtcgccggaattcagcgacgccggggcccgcgaggcctggatgtccgactccaccctggtcccgatccgggaagtgatcgagcgcatcgccgccagccaggactgggtggagatcctggtcgccggcacgctcgtcttcgagcctctggtcggccacctggcgaaggccgagttgttcagccgccgtgcgccaatgttcggggacgggaccacgccggcggtgctggcgtcggccctgctggacagcggcaggcacctcgaatcggtccaggcgctcgtccgcctcgtctgccaagaccccgtccatggcgaccagaaccaggcgactgtgcggcggtggatcgaggaatggcagccgcggtgcaaggcggcggcccagtccttcctgccgacgttctccgactgcggcatcgacgccaaggaaagcgccaacgcgctgtcccgggcgctggcgaaccagcgggccgccgtcgagggcgccggcatcacggcatga

SEQ ID NO: 10 - protein, 343 aa, Nocardia corallina
MTTEATVARPVELEGHRTFTWFTPARRKPTEYELYTVGQQSTPDEWLHVDWPLRFDDGRAPWEEESSAVRTSEWSAYRDPHQLWQRPYVSTCNQDQQALARLVPVLTMGSAAITPIWSQKILARSYAAWPFVEYGLFLSLAYAVRQAMSDTVQFSVVFQAVDRMRLLQDIVHHLDHLQESPEFSDAGAREAWMSDSTLVPIREVIERIAASQDWVEILVAGTLVFEPLVGHLAKAELFSRRAPMFGDGTTPAVLASALLDSGRHLESVQALVRLVCQDPVHGDQNQATVRRWIEEWQPRCKAAAQSFLPTFSDCGIDAKESANALSRALANQRAAVEGAGITA

SEQ ID NO: 11 - DNA, 1506 bp, Nocardia corallina
  CDS (1)..(1506)
atggcatcgaaccccacccagctccacgagaagtcgaagtcctacgactgggacttcacctccgtcgagcggcgccccaagttcgagacgaagtacaagatgcccaagaagggcaaggacccgttccgcgtcctgatccgtgactacatgaagatggaagcggagaaggacgaccggacccatggcttcctcgacggcgccgtgcggacgcgtgaggccaccaggattgagccgcggttcgctgaggccatgaagatcatggtgccgcagctgaccaacgccgagtaccaggcggtggcgggctgcggaatgatcatctcggccgtcgagaaccaggagctccgtcagggctacgccgctcagatgctcgatgaggtgcggcacgcgcagctcgagatgacgctacgcaactactacgcgaagcactggtgcgatccctccggcttcgacatcggtcagcgcggcctgtaccagcaccccgcggggctggtgtccatcggcgagttccagcacttcaatactggtgacccgcttgacgtcatcatcgatctcaacatcgtggccgagacggcgttcacgaacatcctgctggtggccactccacaggtcgccgtggccaacggggacaacgcgatggccagcgtgttcctctcgatccagtcggacgaggccaggcacatggccaacgggtacggctcggtcatggcgctgctggagaacgaggacaacctcccgctgctcaaccagtctctcgatcggcacttctggcgtgcccacaaggccttggacaacgcggtcggatggtgttcggagtatggcgcccgcaagcggccatggagctacaaggcccagtgggaggaatgggtcgtcgacgacttcgtgggcggctacatcgaccgactcagcgagttcggcgttcaggctccggcctgccttggcgcggccgccgacgaggtcaagtggtcgcaccacacgctcggtcaggtgctgtcggcggtgtggccgctgaacttctggcgctcggacgccatgggaccggcggacttcgagtggttcgagaaccactacccgggctggagcgcggcctaccagggttactgggagggctacaaggcgctcgccgacccagcaggcggacgcatcatgctccaggagctgccgggtctgccgccgatgtgtcaggtgtgccaggtgccgtgcgtgatgccgcggctggatatgaacgccgcgcggatcatcgagttcgaggggcagaaaatcgcgctgtgcagcgaaccctgccagcggatcttcaccaactggccggaggcgtaccgccaccgcaagcaatactgggcccgctaccacggatgggacctggcggacgtcatcgttgatctcggctacatccgcccggacggcaagaccctcatcggccagccgctgctcgagatggagcggctgtggaccatcgacgacatccgggcccttcagtacgaagtcaaggacccgttgcaggaggcgtga

SEQ ID NO: 12 - protein, 501 aa, Nocardia corallina
MASNPTQLHEKSKSYDWDFTSVERRPKFETKYKMPKKGKDPFRVLIRDYMKMEAEKDDRTHGFLDGAVRTREATRIEPRFAEAMKIMVPQLTNAEYQAVAGCGMIISAVENQELRQGYAAQMLDEVRHAQLEMTLRNYYAKHWCDPSGFDIGQRGLYQHPAGLVSIGEFQHFNTGDPLDVIIDLNIVAETAFTNILLVATPQVAVANGDNAMASVFLSIQSDEARHMANGYGSVMALLENEDNLPLLNQSLDRHFWRAHKALDNAVGWCSEYGARKRPWSYKAQWEEWVVDDFVGGYIDRLSEFGVQAPACLGAAADEVKWSHHTLGQVLSAVWPLNFWRSDAMGPADFEWFENHYPGWSAAYQGYWEGYKALADPAGGRIMLQELPGLPPMCQVCQVPCVMPRLDMNAARIIEFEGQKIALCSEPCQRIFTNWPEAYRHRKQYWARYHGWDLADVIVDLGYIRPDGKTLIGQPLLEMERLWTIDDIRALQYEVKDPLQEA

SEQ ID NO: 13 - DNA, 1494 bp, Xanthobacta sp.
  CDS (1)..(1494)
atggcgctcttgaatcgggacgattggtacgacatcgcgcgcgatgtcgactggacgctcagctatgtcgaccgcgcggtcgcctttcccgaggagtggaaaggcgaaaaggacatttgcggcacggcctgggacgattgggacgagcccttccgggtctccttccgcgaatatgtgatggtccagcgcgacaaggaagcgagcgtcggcgccatccgcgaggccatggtccgcgccaaggcctatgagaagctcgacgacggccacaaggccacctcgcacctgcacatgggcaccatcaccatggtggagcacatggcggtcaccatgcagagccggttcgtgcgcttcgcgccgtccgcccgctggcgcagcctcggggcgttcggcatgctggacgagacccgccacacccagctcgacctgcgcttcagccacgatctgctcaacgattccccgagcttcgactggagccagcgggcgttccacaccgacgaatgggcggttctcgccacccgcaacctgttcgacgacatcatgctcaacgccgactgcgtggaggcggcgctcgccaccagcctgacgctggagcacggcttcaccaacatccagttcgtggcgctcgcctccgacgccatggaagccggcgacgtgaacttctccaacctcttgtcgagcatccagaccgacgaggcgcggcacgcccagttgggctttcccaccctcgacgtgatgatgaagcacgaccccaagcgcgcccagcagatcctggacgtcgccttctggcgctcctaccgcatcttccaggcggtgaccggcgtctccatggactactacacgccggtcgccaagcggcagatgtcgttcaaggagttcatgctggagtggatcgtcaagcatcatgagcgcatcctgcgcgactacggcctccagaagccctggtactgggacacgttcgagaagaccctcgatcacggccaccacgcgctgcacatcggcacctggttctggcgcccgaccctgttctgggatcccaatggcggcgtctcgcgcgaggagcggcgctggctgaaccagaagtatccgaactgggaagagagctggggcgtcctgtgggacgagatcatctccaacatcaatgcgggcaacattgaaaagaccttgcccgagacgctgccgatgctgtgcaacgtcaccaacctgcccatcggctcgcactgggaccgcttccacctgaagcccgagcagctcgtctacaaggggcggctctacaccttcgacagcgacgtctccaagtggatcttcgagctcgatccggagcgctatgccggccacaccaacgtggtcgaccgcttcatcggcgggcagatccagcccatgaccatcgagggcgtgctcaactggatgggcctgacgcccgaagtcatgggcaaggacgtgttcaactaccgttgggccggcgattacgccgagaaccggatcgccgccgagtaa

SEQ ID NO: 14 - protein, 497 aa, Xanthobacta sp.
MALLNRDDWYDIARDVDWTLSYVDRAVAFPEEWKGEKDICGTAWDDWDEPFRVSFREYVMVQRDKEASVGAIREAMVRAKAYEKLDDGHKATSHLHMGTITMVEHMAVTMQSRFVRFAPSARWRSLGAFGMLDETRHTQLDLRFSHDLLNDSPSFDWSQRAFHTDEWAVLATRNLFDDIMLNADCVEAALATSLTLEHGFTNIQFVALASDAMEAGDVNFSNLLSSIQTDEARHAQLGFPTLDVMMKHDPKRAQQILDVAFWRSYRIFQAVTGVSMDYYTPVAKRQMSFKEFMLEWIVKHHERILRDYGLQKPWYWDTFEKTLDHGHHALHIGTWFWRPTLFWDPNGGVSREERRWLNQKYPNWEESWGVLWDEIISNINAGNIEKTLPETLPMLCNVTNLPIGSHWDRFHLKPEQLVYKGRLYTFDSDVSKWIFELDPERYAGHTNVVDRFIGGQIQPMTIEGVLNWMGLTPEVMGKDVFNYRWAGDYAENRIAAE

SEQ ID NO: 15 - DNA, 1026 bp, Xanthobacta sp.
  CDS (1)..(1026)
atgacacagcagcgccccacccgcacgcgcgagcgcaagaagacctggacggctttcggcaatctcggacgcaagccgaccgactacgaggtcgtcacccacaacatgaaccacaccatgcgcggcacgcccctggagctgtcgccgacggtgcacgccaatgtgtggctcaagaagaaccgcgacgagatcgcgctcaaggtcgacagctgggatctgttccgcgatcccgaccgcaccacctacgacacctacgtcaagatgcaggacgaccaggagacctatgtcgacaacctgctcctgtcctacaccggcgagggccgctacgacgaggagctttcctcgcgcagcctcgacctcctgtccgcggggctgacgccgacccgctatctgggccatgggctgcagatgctcgcggcctatatccagcagctcgccccgtcggcctatgtgggcaattgcgcggtgttccagacctccgacgcgctgcgccgcgtgcagcgcgtcgcctaccgcacccgccagctcgccgacgcccatccggcccgcggcttcggctccggcgaccgggcggtgtgggagaagtccccggactggcagcccatccgcaaggccatcgaggagctgctcgtcaccttcgaatgggacaaggcgctcgccggcaccaatttcgtggtgaagccgatcctcgacgagctgttcctcaaccacctggcgcgcctgctccacgtggagggcgacgagctcgacagcctcgtgctgcggaaccttcacggcgacgcccagcgccacgcccgctggacggccgcgctcggccgcttcgccgtcgagcagaacgtgaacaaccgcacggtcctgcgcgacgccatcgccggctggcacgagaccggcgaggcggtcctcgccgcgggcgccgggatgcttgcgagccgcgcccccagcgcggatgcggccaagatcgccgacgaggtccgcgccacgctcgcgcagctgcacgccaatgcgggcctcgggcacgatgcctga

SEQ ID NO: 16 - protein, 341 aa, Xanthobacta sp.
MTQQRPTRTRERKKTWTAFGNLGRKPTDYEVVTHNMNHTMRGTPLELSPTVHANVWLKKNRDEIALKVDSWDLFRDPDRTTYDTYVKMQDDQETYVDNLLLSYTGEGRYDEELSSRSLDLLSAGLTPTRYLGHGLQMLAAYIQQLAPSAYVGNCAVFQTSDALRRVQRVAYRTRQLADAHPARGFGSGDRAVWEKSPDWQPIRKAIEELLVTFEWDKALAGTNFVVKPILDELFLNHLARLLHVEGDELDSLVLRNLHGDAQRHARWTAALGRFAVEQNVNNRTVLRDAIAGWHETGEAVLAAGAGMLASRAPSADAAKIADEVRATLAQLHANAGLGHDA

SEQ ID NO: 17 - DNA, 267 bp, Xanthobacta sp.
  CDS (1)..(267)
atgtctttgttccccatcgtgggccgcttcgtgggggatttcgtcccccacctggtggcggtggacacctctgacaccatcgatcagatcgccgagaaggtggcggtccacacggtcgggcggcgcttgccgcccgatcccaccgccaccggctatgaggtgctcctcgacggcgagaccctggacgggggcgccaccctggaggccatcatgaccaagcgcgagatgctgcccctgcagtggttcgacgtgaggttcaagaagtga

SEQ ID NO: 18 - protein, 88 aa, Xanthobacta sp.
MSLFPIVGRFVGDFVPHLVAVDTSDTIDQIAEKVAVHTVGRRLPPDPTATGYEVLLDGETLDGGATLEAIMTKREMLPLQWFDVRFKK

SEQ ID NO: 19 - DNA, 1584 bp, Methylococcus capsulatas
  CDS (1)..(1584)
atggcacttagcaccgcaaccaaggccgcgacggacgcgctggctgccaatcgggcacccaccagcgtgaatgcacaggaagtgcaccgttggctccagagcttcaactgggatttcaagaacaaccggaccaagtacgccaccaagtacaagatggcgaacgagaccaaggaacagttcaagctgatcgccaaggaatatgcgcgcatggaggcagtcaaggacgaaaggcagttcggtagcctgcaggatgcgctgacccgcctcaacgccggtgttcgcgttcatccgaagtggaacgagaccatgaaagtggtttcgaacttcctggaagtgggcgaatacaacgccatcgccgctaccgggatgctgtgggattccgcccaggcggcggaacagaagaacggctatctggcccaggtgttggatgaaatccgccacacccaccagtgtgcctacgtcaactactacttcgcgaagaacggccaggacccggccggtcacaacgatgctcgccgcacccgtaccatcggtccgctgtggaagggcatgaagcgcgtgttttccgacggcttcatttccggcgacgccgtggaatgctccctcaacctgcagctggtgggtgaggcctgcttcaccaatccgctgatcgtcgcagtgaccgaatgggctgccgccaacggcgatgaaatcaccccgacggtgttcctgtcgatcgagaccgacgaactgcgccacatggccaacggttaccagaccgtcgtttccatcgccaacgatccggcttccgccaagtatctcaacacggacctgaacaacgccttctggacccagcagaagtacttcacgccggtgttgggcatgctgttcgagtatggctccaagttcaaggtcgagccgtgggtcaagacgtgggaccgctgggtgtacgaggactggggcggcatctggatcggccgtctgggcaagtacggggtggagtcgccgcgcagcctcaaggacgccaagcaggacgcttactgggctcaccacgacctgtatctgctggcttatgcgctgtggccgaccggcttcttccgtctggcgctgccggatcaggaagaaatggagtggttcgaggccaactaccccggctggtacgaccactacggcaagatctacgaggaatggcgcgcccgcggttgcgaggatccgtcctcgggcttcatcccgctgatgtggttcatcgaaaacaaccatcccatctacatcgatcgcgtgtcgcaagtgccgttctgcccgagcttggccaagggcgccagcaccctgcgcgtgcacgagtacaacggcgagatgcacaccttcagcgaccagtggggcgagcgcatgtggctggccgagccggagcgctacgagtgccagaacatcttcgaacagtacgaaggacgcgaactgtcggaagtgatcgccgaactgcacgggctgcgcagtgatggcaagaccctgatcgcccagccgcatgtccgtggcgacaagctgtggacgttggacgatatcaaacgcctgaactgcgtcttcaagaacccggtgaaggcattcaattga

SEQ ID NO: 20 - protein, 527 aa, Methylococcus capsulatas
MALSTATKAATDALAANRAPTSVNAQEVHRWLQSFNWDFKNNRTKYATKYKMANETKEQFKLIAKEYARMEAVKDERQFGSLQDALTRLNAGVRVHPKWNETMKVVSNFLEVGEYNAIAATGMLWDSAQAAEQKNGYLAQVLDEIRHTHQCAYVNYYFAKNGQDPAGHNDARRTRTIGPLWKGMKRVFSDGFISGDAVECSLNLQLVGEACFTNPLIVAVTEWAAANGDEITPTVFLSIETDELRHMANGYQTVVSIANDPASAKYLNTDLNNAFWTQQKYFTPVLGMLFEYGSKFKVEPWVKTWDRWVYEDWGGIWIGRLGKYGVESPRSLKDAKQDAYWAHHDLYLLAYALWPTGFFRLALPDQEEMEWFEANYPGWYDHYGKIYEEWRARGCEDPSSGFIPLMWFIENNHPIYIDRVSQVPFCPSLAKGASTLRVHEYNGEMHTFSDQWGERMWLAEPERYECQNIFEQYEGRELSEVIAELHGLRSDGKTLIAQPHVRGDKLWTLDDIKRLNCVFKNPVKAFN

SEQ ID NO: 21 - DNA, 1170 bp, Methylococcus capsulatas
  CDS (1)..(1170)
atgagcatgttaggagaaagacgccgcggtctgaccgatccggaaatggcggccgtcattttgaaggcgcttcctgaagctccgctggacggcaacaacaagatgggttatttcgtcaccccccgctggaaacgcttgacggaatatgaagccctgaccgtttatgcgcagcccaacgccgactggatcgccggcggcctggactggggcgactggacccagaaattccacggcggccgcccttcctggggcaacgagaccacggagctgcgcaccgtcgactggttcaagcaccgtgacccgctccgccgttggcatgcgccgtacgtcaaggacaaggccgaggaatggcgctacaccgaccgcttcctgcagggttactccgccgacggtcagatccgggcgatgaacccgacctggcgggacgagttcatcaaccggtattggggcgccttcctgttcaacgaatacggattgttcaacgctcattcgcagggcgcccgggaggcgctgtcggacgtaacccgcgtcagcctggctttctggggcttcgacaagatcgacatcgcccagatgatccaactcgaacggggtttcctcgccaagatcgtacccggtttcgacgagtccacagcggtgccgaaggccgaatggacgaacggggaggtctacaagagcgcccgtctggccgtggaagggctgtggcaggaggtgttcgactggaacgagagcgctttctcggtgcacgccgtctatgacgcgctgttcggtcagttcgtccgccgcgagttctttcagcggctggctccccgcttcggcgacaatctgacgccattcttcatcaaccaggcccagacatacttccagatcgccaagcagggcgtacaggatctgtattacaactgtctgggtgacgatccggagttcagcgattacaaccgtaccgtgatgcgcaactggaccggcaagtggctggagcccacgatcgccgctctgcgcgacttcatggggctgtttgcgaagctgccggcgggcaccactgacaaggaagaaatcaccgcgtccctgtaccgggtggtcgacgactggatcgaggactacgccagcgcgatcgacttcaaggcggaccgcgatcagatcgttaaagcggttctggcaggattgaaataa

SEQ ID NO: 22 - protein, 389 aa, Methylococcus capsulatas
MSMLGERRRGLTDPEMAAVILKALPEAPLDGNNKMGYFVTPRWKRLTEYEALTVYAQPNADWIAGGLDWGDWTQKFHGGRPSWGNETTELRTVDWFKHRDPLRRWHAPYVKDKAEEWRYTDRFLQGYSADGQIRAMNPTWRDEFINRYWGAFLFNEYGLFNAHSQGAREALSDVTRVSLAFWGFDKIDIAQMIQLERGFLAKIVPGFDESTAVPKAEWTNGEVYKSARLAVEGLWQEVFDWNESAFSVHAVYDALFGQFVRREFFQRLAPRFGDNLTPFFINQAQTYFQIAKQGVQDLYYNCLGDDPEFSDYNRTVMRNWTGKWLEPTIAALRDFMGLFAKLPAGTTDKEEITASLYRVVDDWIEDYASAIDFKADRDQIVKAVLAGLK

SEQ ID NO: 23 - DNA, 513 bp, Methylococcus capsulatas
  CDS (1)..(513)
atggcgaaactgggtatacacagcaacgacacccgcgacgcctgggtgaacaagatcgcgcagctcaacaccctggaaaaagcggccgagatgctgaagcagttccggatggaccacaccacgccgttccgcaacagctacgaactggacaacgactacctctggatcgaggccaagctcgaagagaaggtcgccgtcctcaaggcacgcgccttcaacgaggtggacttccgtcataagaccgctttcggcgaggatgccaagtccgttctggacggcaccgtcgcgaagatgaacgcggccaaggacaagtgggaggcggagaagatccatatcggtttccgccaggcctacaagccgccgatcatgccggtgaactatttcctggacggcgagcgtcagttggggacccggctgatggaactgcgcaacctcaactactacgacacgccgctggaagaactgcgcaaacagcgcggtgtgcgggtggtgcatctgcagtcgccgcactga

SEQ ID NO: 24 - protein, 170 aa, Methylococcus capsulatas
MAKLGIHSNDTRDAWVNKIAQLNTLEKAAEMLKQFRMDHTTPFRNSYELDNDYLWIEAKLEEKVAVLKARAFNEVDFRHKTAFGEDAKSVLDGTVAKMNAAKDKWEAEKIHIGFRQAYKPPIMPVNYFLDGERQLGTRLMELRNLNYYDTPLEELRKQRGVRVVHLQSPH

SEQ ID NO: 25 - DNA, 1206 bp, Pseudomonas oleovorans
  CDS (1)..(1206)
atgcttgagaaacacagagttctggattccgctccagagtacgtagataaaaagaaatatctctggatactatcaactttgtggccggctactccgatgatcggaatctggcttgcaaatgaaactggttgggggattttttatgggctggtattgctcgtatggtacggcgcacttccattgcttgatgcgatgtttggtgaggactttaataatccgcctgaagaagtggtgccgaaactagagaaggagcggtactatcgagttttgacatatctaacagttcctatgcattacgctgcattaattgtgtcagcatggtgggtcggaactcagccaatgtcttggcttgaaattggtgcgcttgccttgtcactgggtatcgtgaacggactagcgctcaatacaggacacgaactcggtcacaagaaggagacttttgatcgttggatggccaaaattgtgttggctgtcgtagggtacggtcacttctttattgagcataataagggtcatcaccgtgatgtcgctacaccgatggatcctgcaacatcccggatgggagaaagcatttataagttttcaatccgtgagatcccaggagcatttattcgtgcttgggggcttgaggaacaacgcctttcgcgccgtggccaaagcgtttggagtttcgataatgaaatcctccaaccaatgatcatcacagttattctttacgccgttctccttgccttgtttggacctaagatgctggtgttcctgccgattcaaatggctttcggttggtggcagctgaccagtgcgaactatattgaacattacggcttgctccgtcaaaaaatggaggacggtcgatatgagcatcaaaagccgcaccattcttggaatagtaatcacatcgtctctaatctagtgctgttccaccttcagcggcactcggatcaccacgcgcatccaacacgttcttatcagtcacttcgggattttcccggcctgccggctcttccgacgggttaccctggtgcatttttgatggcgatgattcctcagtggtttagatcagttatggatcccaaggtagtagattgggctggtggtgaccttaataagatccaaattgatgattcgatgcgagaaacctatttgaaaaaatttggcactagtagtgctggtcatagttcgagtacctctgcggtagcatcgtag

SEQ ID NO: 26 - protein, 401 aa, Pseudomonas oleovorans
MLEKHRVLDSAPEYVDKKKYLWILSTLWPATPMIGIWLANETGWGIFYGLVLLVWYGALPLLDAMFGEDFNNPPEEVVPKLEKERYYRVLTYLTVPMHYAALIVSAWWVGTQPMSWLEIGALALSLGIVNGLALNTGHELGHKKETFDRWMAKIVLAVVGYGHFFIEHNKGHHRDVATPMDPATSRMGESIYKFSIREIPGAFIRAWGLEEQRLSRRGQSVWSFDNEILQPMIITVILYAVLLALFGPKMLVFLPIQMAFGWWQLTSANYIEHYGLLRQKMEDGRYEHQKPHHSWNSNHIVSNLVLFHLQRHSDHHAHPTRSYQSLRDFPGLPALPTGYPGAFLMAMIPQWFRSVMDPKVVDWAGGDLNKIQIDDSMRETYLKKFGTSSAGHSSSTSAVAS

SEQ ID NO: 27 - DNA, 1560 bp, Burkholderia cepacia
  CDS (1)..(1560)
atggacacttctgtgcagaagaagaaactcggtttaaagaatcgctacgcagcgatgacccgcggtcttggctggcagaccagctaccagccgatggagaaagtgtttccgtacgacaagtacgaaggcatcaagatccacgattgggataaatgggaagaccccttccgcctgaccatggacgcctactggaaatatcagggcgagaaggaaaaaaagctttacgccgtcatcgacgctttcgcgcagaacaacgggcagttgagcatttccgacgcgcgatatgtcaacgcactcaaggtgtttatccagggtgtgacaccgttggagtatatggcacaccgaggttttgcccacattggtcggcattttacgggtgaaggggcacgtgttgcttgccagatgcagtccatcgacgagctgcgtcacttccagaccgaaatgcatgctctctcgcactacaacaagtattttaacggtctgcacaactccatccattggtacgaccgggtttggtatttgtcggtgcccaagtcattttttgaagacgcggccaccggtggaccgttcgagtttcttaccgcggtgagcttttcgttcgaatatgtgttgaccaacctgctgtttgtccccttcatgtcgggtgctgcttacaacggggacatgtctacggtcactttcggtttttcggcgcaaagtgacgaatcgcgccacatgacactcggcatcgaatgcatcaagttcatgctagaacaggatccggacaacgtgcccatcgtgcagcgctggatcgacaagtggttctggcgcggctatcggctgttgagcatcgtggccatgatgcaggactacatgctgcccaaccgggtgatgagctggcgcgagagctgggagatgtacgtcgagcagaacggcggcgcgctgttcaaggatcttgcgcgttatggcatccgcaagcccaagggctgggaccaggcttgcgaaggcaaggaccacatcagccatcagaccttcgccgtattctataactataacgccgcggcccccatccacacctgggttcccacaaaagaagaaatgggatggctgtcggagaagtaccccgagacgttcgacaagtattaccgtccgcgttgggactactggcgcgagcaggccgccaagggcaaccgtttctacaacaagacgctgccgatgctctgcactacctgccagattccgatgatattcaccgagcctggcgacgcaaccaagatctgctatcgcgagtcggcctacctcggcgacaagtatcacttctgcagcgaccactgcaaggagatttttgacaacgaacccgaaaagttcgtgcagtcatggcttccgccgcagcaagtgtatcaaggaaactgtttcaagccggatgccgatccgaccaaggagggttttgatcccttgatggccttgctcgactactacaacctgaatgtaggccgggacaacttcgatttcgagggatcggaagaccaaaagaactttgctgcctggcgtggagaggtcttgcaaggagaagccaaatga

SEQ ID NO: 28 - protein, 519 aa, Burkholderia cepacia
MDTSVQKKKLGLKNRYAAMTRGLGWQTSYQPMEKVFPYDKYEGIKIHDWDKWEDPFRLTMDAYWKYQGEKEKKLYAVIDAFAQNNGQLSISDARYVNALKVFIQGVTPLEYMAHRGFAHIGRHFTGEGARVACQMQSIDELRHFQTEMHALSHYNKYFNGLHNSIHWYDRVWYLSVPKSFFEDAATGGPFEFLTAVSFSFEYVLTNLLFVPFMSGAAYNGDMSTVTFGFSAQSDESRHMTLGIECIKFMLEQDPDNVPIVQRWIDKWFWRGYRLLSIVAMMQDYMLPNRVMSWRESWEMYVEQNGGALFKDLARYGIRKPKGWDQACEGKDHISHQTFAVFYNYNAAAPIHTWVPTKEEMGWLSEKYPETFDKYYRPRWDYWREQAAKGNRFYNKTLPMLCTTCQIPMIFTEPGDATKICYRESAYLGDKYHFCSDHCKEIFDNEPEKFVQSWLPPQQVYQGNCFKPDADPTKEGFDPLMALLDYYNLNVGRDNFDFEGSEDQKNFAAWRGEVLQGEAK

SEQ ID NO: 29 - DNA, 996 bp, Burkholderia cepacia
  CDS (1)..(996)
atgaccatcgatttgaagacgcgggaaatcaaaccactgcgtcacacctacacgcacgtggctcaatacatcggggccgataaagccgcttcgcgctatcaggaaggcactgtaggtgctcaacccgcagcgaattttcattaccggcccacgtgggatcccgagcatgaactgttcgacacgtcgcgtaccgcgattcaaatgaaggactggtatgcgctgaaagacccgcgtcagttctactacgcgtcgtggacgatgacccgagcgcggcagcaagacgcgatggaatccaacttcgagtttgtcgagtcgcgcggcatgatcgatctcgtttccgatgaggttcgacaacgggcgctttccgttctcgtgcctttgcgtcacgcggcctggggcgcgaacatgaacaactcccagatctgtgccctaggttatggcacgaccttcactgcgccggctatgttccacgcaatggacaatctgggtgtagcgcagtatctcacacgactggcgctggtaatgtctggacccgatcttcttgacgaagccaagcaagcctggatgacgagtcccgattggcaaccgttgcgtcgttatgtggaaaacactctggtgctgcaagatccggtggaactgttcatcgcccaaaatctggcgctcgacggtcttctttatcccatgatctacggcgctttcgtcgacgattacatcgcactcaacggtggtagcgcagtggcaatgctaaccactttcatgcccgagtggcatgacgaatccagtcgctgggtcgatgcggtagtaaagaccatggcgacggaatcggaggataacaaagcgctgctcattcactggttgcgtacctgggaagatcaggcggcgtcagcgttgttgcctgtcgctgaaatggctttggcggaaaacggccacgacgccttggaagaagtaaggcagcaacttcgtgcccgcgttgcgaaggccgggattgttctgtaa

SEQ ID NO: 30 - protein, 331 aa, Burkholderia cepacia
MTIDLKTREIKPLRHTYTHVAQYIGADKAASRYQEGTVGAQPAANFHYRPTWDPEHELFDTSRTAIQMKDWYALKDPRQFYYASWTMTRARQQDAMESNFEFVESRGMIDLVSDEVRQRALSVLVPLRHAAWGANMNNSQICALGYGTTFTAPAMFHAMDNLGVAQYLTRLALVMSGPDLLDEAKQAWMTSPDWQPLRRYVENTLVLQDPVELFIAQNLALDGLLYPMIYGAFVDDYIALNGGSAVAMLTTFMPEWHDESSRWVDAVVKTMATESEDNKALLIHWLRTWEDQAASALLPVAEMALAENGHDALEEVRQQLRARVAKAGIVL

SEQ ID NO: 31 - DNA, 357 bp, Burkholderia cepacia
  CDS (1)..(357)
atgagcgttgttgccctcaaaccctacaagttcccggcacgagacgcgcgcgaaaactttccggcgccgttgctgtttatcggctgggaagaccatctgttgtttgcggcacctgttgccttgcccctgccgtcggacacgttgttcggtgcgctgtgcacccaggtgttgcccggcacttatggctatcaccccgatttctcaaagatcgactggagccaggtgcagtggtttaagtccggccagccgtggcatcccgacccggcgaagtcgctggctgaaaacggtctgacgcacaaagacgtgatccgctttcgcacgcctggcttgaacggtctgagcggttcctgcaattga

SEQ ID NO: 32 - protein, 118 aa, Burkholderia cepacia
MSVVALKPYKFPARDARENFPAPLLFIGWEDHLLFAAPVALPLPSDTLFGALCTQVLPGTYGYHPDFSKIDWSQVQWFKSGQPWHPDPAKSLAENGLTHKDVIRFRTPGLNGLSGSCN

SEQ ID NO: 33 - DNA, 1143 bp, Bacillus stearothermophilus
  CDS (1)..(1143)
atggaaaaaaataaaatgttaatagaagaaaagttggacactgctgctcttcttgctaaggcggaggaaataggccggattgctgaggaagaggcgggtgaagcggaccgcaatgcctgtttctccgaccgggtggctagggccattaaagaagctggattccacaagctcatgcgtcccaagcagtacggaggactgcaagtagacttgcgaacttacggggagattgtccgcacagtggcccggtacagtgttgccgcaggatggctgacctatttttattccatgcatgaggtttgggctgcatatctgcctccaaaaggcagagaagaaatttttggacaaggagggctgttggcagacgtcgttgcccctgttggccgggtggagaaggacggggacggctaccgtctctatgggcagtggaacttctgtagcggtgtcctccatagtgactggatcggacttggcgccatgatggagctgcctgacggcaatagtcctgagtactgtttgttagtgctgcctaagtcggatgtccagatcgtagaaaattgggataccatgggcctccgcgcttcgggaagcaacggggtattagttgaaggtgcttatgttccattacaccggatctttccggctggccgggtgatggctcatccgcttttcttgcttgggttccctttagtatctttaggcggcgacgaacgattggtgtcacttttccaagaacgcactgagaagcgcattcgtgtcttcaaaggcggcgcgaaagaaaaggattctgccgctagccagcggctgttagccgagatgaaaacagaattaaatgcaatggaaggcattgtggaacaatatatccgccagcttgaggcttgccaaaaagaaggaaagacggtgatgaacgatatggagcgagagcagctattcgcatggcgtggatatgtggcaaaagcgtccgccaatattgccgtcagaacactgttaactcttggaggcaattcgatctttaaaggcgatccggtagaactgttcacaagagatttgctagcggtggccgcacatcctaactccctgtgggaggatgcgatggctgcatatggaagaacgatattcgggctgccaggggacccagtctggtaa

SEQ ID NO: 34 - protein, 380 aa, Bacillus stearothermophilus
MEKNKMLIEEKLDTAALLAKAEEIGRIAEEEAGEADRNACFSDRVARAIKEAGFHKLMRPKQYGGLQVDLRTYGEIVRTVARYSVAAGWLTYFYSMHEVWAAYLPPKGREEIFGQGGLLADVVAPVGRVEKDGDGYRLYGQWNFCSGVLHSDWIGLGAMMELPDGNSPEYCLLVLPKSDVQIVENWDTMGLRASGSNGVLVEGAYVPLHRIFPAGRVMAHPLFLLGFPLVSLGGDERLVSLFQERTEKRIRVFKGGAKEKDSAASQRLLAEMKTELNAMEGIVEQYIRQLEACQKEGKTVMNDMEREQLFAWRGYVAKASANIAVRTLLTLGGNSIFKGDPVELFTRDLLAVAAHPNSLWEDAMAAYGRTIFGLPGDPVW

SEQ ID NO: 35 - DNA, 1191 bp, Helianthus annuus
  CDS (1)..(1191)
atggcgattcgcatcaatacggcgacgtttcaatcagacctgtaccgttcattcgcgtttcctcaaccgaaacctctcagatctcccaaattcgccatggcttccaccattggatccgctacaacgaaggttgaaagcaccaaaaagccctttacccctccaagggaggttcaccaacaggtgctacactcaatgccgccacaaaagatcgaaatcttcaaatccatggagggttgggccgaaaataacatattggttcacctaaagcctgtcgaaaaatgctggcaagcacaggatttcctaccagatcccgcatctgacggatttatggaacaagtggaggaattacgggctcgggctaaggagattccggatgattactttgttgttttggttggagatatgattactgaagaagcactgcctacttaccaaacaatgcttaatactcttgatggtgtgcgtgatgagaccggggctaccctacttcttgggctagtctggactcgggcttggaccgctgaagaaaacaggcacggtgatcttctacatcagtatctgtatcttagtgggcgggtcgacatgaggcagattcagaagacaattcagtacctcattgggtctggaatggacccccggaccgaaaacagtccttaccttgggttcatctacacttcatttcaagagcgtgccaccttcatctctcacggaaacacagcccggcacgcaaaggagcatggtgacgtgaagctggctcaaatgtgcggtataattgcagctgatgaaaaaaggcacgaaaccgcctacacaaaaatagtagaaaaactcttcgaaattgacccggacggcactgttctcgcttttgccgacatgatgaggaaaaagatctccatgcctgcacacttgatgtacgatgggcgtgatgataacctcttcgaaaatttctcagctgttgcccaaaggctcggtgtgtacactgcgaaggactatgcagacattctggagtttctggtgggccggtggaaggtggcggatttaaccgggctttctggtgaagggcgtaaagcccaagactatgtgtgcgggctggccccaagaatcagaaggcttgaggagaggaactcggcaagggcgaaggaaagtgtgaacgttccgttcagctggatctttgatagagaagtgaagctctga

SEQ ID NO: 36 - protein, 396 aa, Helianthus annuus
MAIRINTATFQSDLYRSFAFPQPKPLRSPKFAMASTIGSATTKVESTKKPFTPPREVHQQVLHSMPPQKIEIFKSMEGWAENNILVHLKPVEKCWQAQDFLPDPASDGFMEQVEELRARAKEIPDDYFVVLVGDMITEEALPTYQTMLNTLDGVRDETGATLLLGLVWTRAWTAEENRHGDLLHQYLYLSGRVDMRQIQKTIQYLIGSGMDPRTENSPYLGFIYTSFQERATFISHGNTARHAKEHGDVKLAQMCGIIAADEKRHETAYTKIVEKLFEIDPDGTVLAFADMMRKKISMPAHLMYDGRDDNLFENFSAVAQRLGVYTAKDYADILEFLVGRWKVADLTGLSGEGRKAQDYVCGLAPRIRRLEERNSARAKESVNVPFSWIFDREVKL